(19) United States
(12) Patent Application Publication
(10) Pub. No.: US 2023/0248637 A1
(43) Pub. Date: Aug. 10, 2023

(54) HAIR PROTECTION AND REPAIR COMPOSITION

(71) Applicant: Barnet Products LLC, Englewood Cliffs, NJ (US)

(72) Inventor: Walter Smith, Wellington, FL (US)

(73) Assignee: Barnet Products LLC, Englewood Cliffs, NJ (US)

(21) Appl. No.: 18/107,207

(22) Filed: Feb. 8, 2023

Related U.S. Application Data
(60) Provisional application No. 63/307,711, filed on Feb. 8, 2022.

(51) Int. Cl. A61K 8/92; A61Q 5/00; A61Q 5/12

(52) U.S. Cl. CPC A61K 8/922; A61Q 5/002; A61Q 5/12; A61K 2800/5922

(57) ABSTRACT

An oil-based composition for use in a rinse off or leave on hair product for protecting hair cuticles, the composition including a triglyceride carrier, squalene, a coconut oil-based alkane, melon seed oil, argan seed oil, and argan seed oil unsaponifiables. Using 0.001% to 25% of the composition in a rinse off/leave on product provides many improvements in the condition of hair, such as improving the hair moisture content; hair shine; reduction in flyaways; reduction in inter fiber friction; improved curl retention; improved hair smoothness; fill in/correction of hair cuticle defects; improved appearance of hair: increased hair break strength; and/or hair elasticity. The use of the 0.001%-25% of the composition can also prevent: the loss of hair moisture; the formation of split ends; the formation of cuticle gaps and voids; the loss of hair tensile strength; the loss of hair elasticity; hair breakage; and/or increase inter fiber friction.

This application claims priority to U.S. Provisional Patent Application No 63/307,711 filed Feb. 8, 2022, entitled “HAIR PROTECTION AND REPAIR COMPOSITION,” which is hereby incorporated by reference in its entirety.

The present invention provides an oil-based composition and method of use in a rinse off or leave on hair produce for preventing damage to and repairing damage of the hair cuticle, thus improving the appearance and structural properties of hair.

### SUMMARY OF THE INVENTION

The present invention provides a composition and method for an oil-based composition used in a rinse off or leave on hair product for repairing and protecting the hair cuticle from damage.

The present invention provides an oil-based composition for use in a rinse off or leave on hair product for protecting hair cuticles, the composition including a triglyceride carrier, squalene, a coconut oil-based alkane, melon seed oil, argan seed oil, and argan seed oil unsaponifiables.

### DETAILED DESCRIPTION

The present invention provides a composition that can be used in a rinse off hair product such as a shampoo, conditioner, or a treatment pack or a leave on hair product. Using 0.001% to 25% of the composition of the present invention in a rinse off/leave on product provides many improvements in the condition of hair. Such improvements include: improving the hair moisture content; hair shine; reduction in flyaways; reduction in inter fiber friction which improves hair compatibility; reduction in friction between hair fibers; improved curl retention; improved hair smoothness; fill in/correction of hair cuticle defects: improved appearance of hair; increased hair break strength; and/or hair elasticity. The use of the 0.001%-25% of the composition of the present invention in a rinse off/leave on product can also prevent: the loss of hair moisture; the formation of split ends; the formation of cuticle gaps and voids; the loss of hair tensile strength; the loss of hair elasticity; hair breakage; and/or increase inter fiber friction. The composition includes a triglyceride carrier, squalene, a coconut oil-based alkane, melon seed oil, argan seed oil, and argan seed oil unsaponifiables.

A combination of a polar lipid fraction from argan oil combined with melon oil in a penetration enhancing vehicle provides superior protection against hair damage from a repeated washing/blow drying/combing cycle. The present invention, referred to as CapiSeal™ herein, provides significant hair damage preventing ability. A preferred range of CapiSeal concentration is 1% to 5%, with an optimal concentration from a cost, efficacy, and aesthetics perspective being 30%.

### Experimental Results

### Parameter Assessed

The hair break strength with a mini Instron type tester was evaluated.

### Treatment

Individual Caucasian hair strands, virgin and bleached, were tested for break strength. All tests were completed on a minimum of 10 hair strands. All test cells were subjected to the following hair damaging protocol: wash for 5 minutes at 50° C.-60° C. water with 15% SLS: blow dry 5 min at high heat with a 1000 W hair dryer with combing. This cycle was repeated 10 times

### Test Cells

The following formulas were tested for efficacy. The variations examined a number of compositional parameters including the importance of the argan concentrate, the specificity, concentration and need for Vegelite, the specificity of the carrier triglyceride based oil, and the role of squalane in determining product efficacy.

TABLE 1
CapiSeal ™ Variations

| | Component | | | | | | | | | |
|---|---|---|---|---|---|---|---|---|---|---|
| | % | % | % | % | % | % | % | % | % | % |
| Formula # | 1 | 2 | 3 | 4 | 5 | 6 | 7 | 8 | 9 | 10 |
| Safflower oil | 83.9 | 84 | 93.9 | 83.95 | 83 | 83.5 | 73.9 | 83.9* | 83.9 | 83.9 |
| Vegelite 912 | 10 | 10 | 0 | 10 | 10 | 10 | 20 | 10 | 10** | 14 |
| Squalane | 4 | 4 | 4 | 4 | 4 | 4 | 4 | 4 | 4 | 0 |
| Melon oil | 1 | 1 | 1 | 1 | 1 | 1 | 1 | 1 | 1 | 1 |
| Argan oil | 1 | 1 | 1 | 1 | 1 | 1 | 1 | 1 | 1 | 1 |
| Argan conc | 0.1 | 0 | 0.1 | 0.05 | 1 | 0.5 | 0.1 | 0.1 | 0.1 | 0.1 |

*Sunflower oil
**Hemi squalene

### Test Results

The above compositions, herein referred to as CapiSeal™ variations. were used at 1%, 3% and 5% in a 15% SLS based shampoo. The hair samples were treated as described above. Break strengths from the various tests are listed in the Table 2 below. The results presented are averages from 10 strands tested.

### Vegelite and Triglyceride Oil Requirement and Specificity

The treatment protocol resulted in considerable damage to both virgin and bleached hair as seen in Table 2 in the no treatment cell (25% and 43% respectively). Table 2 (cells 1, 3, 7, 8, 9) also shows that Vegelite 912 (a blend of low molecular weight alkanes) enhances the activity of the protective oils. This is due to its penetration enhancing effects. Removal of Vegelite (cell 3), or substitution for it with an alternative alkane (such as hemi-squalane, Centerchem, or cell 9) results in an inferior product, with some, but not maximal activity. Increasing the concentration of Vegelite to 20% added no additional benefits. Comparing cells 1 and 8 demonstrates that both Sunflower and Safflower oil are equally acceptable from a damage preventing perspective.

TABLE 2
Change in Break Strength

| | | Test Formula Number | | | | | | | | | |
|---|---|---|---|---|---|---|---|---|---|---|---|
| Treatment | None | 1 | 2 | 3 | 4 | 5 | 6 | 7 | 8 | 9 | 10 |
| Virgin | −25% | −6% | −21% | −15% | −10% | 5% | −9% | −7% | −9% | −16% | −14% |
| Bleached | −43% | −4% | −32% | −21% | −12% | −4% | −7% | −8% | −7% | −18% | −14% |
| Argan conc. | | 0.1 | 0 | 0.1 | 0.05 | 1 | 0.5 | 0.1 | 0.1 | 0.1 | 0.1 |
| Vegelite 912 | | 10% | | 0% | | | | 20% | 10% | 10%** | |

To each test formula, CapiSeal ™ was added at 3% to the SLS-based shampoo system.
**Refers to 10% of a Vegelite 912 substitute.

### Argan Concentrate Requirement and Concentration

The absolute requirement for the argan concentrate in the CapiSeal™ blend iss demonstrated in the following Table 3. Table 3 shows that as the Argan concentrate was increased in concentration, increasing beneficial effects were observed. In Table 3 the CapiSeal™ variations were used at 3%. Benefits seemed to maximize between 0.1 and 1% argan concentrate.

TABLE 3
Change in Break Strength - Concentration
Dependence-Argan Concentrate

| Argan conc. | Control | 0 | 0.05 | 0.1 | 0.5 | 1.0 |
|---|---|---|---|---|---|---|
| Virgin | −25% | −21% | −17% | −7% | −7% | 5% |
| Bleached | −43% | −32% | −20% | −8% | −6% | −4% |

To each test formula, CapiSeal ™ was added at 3% to the SLS-based shampoo system. Results are the average of at least 10 measurements.

### Argan Concentrate Specificity

The specificity of the argan concentrate (AC) was also looked at. AC is a blend of polyphenols and terpenes. The average molecular weight of the blend is quite low, such as about 250-350. The composition has no nitrogen (such as amines), sulfur (such as sulfates and sulfites) and is composed of carbon, hydrogen and nitrogen. It has a low acid value <10, and specific gravity of about 0.85. Two commercially available terpene or polyphenol blends were chosen and compared activity. Mixed green tea polyphenols (<85% Ministar) and a Mulberry terpenoid extract (Pacific Technology Partners) were tested. These two substitutes were compared to AC in formula #5 from Table 1. As can been seen below in Table 4, the green tea polyphenols had no effect in providing damage protection. The Mulberry blend, which has some similar characteristics to AC, shows a real, but diminished protective effect, compared to AC.

TABLE 4
Change in Break Strength-
Argan Concentrate specificity

| | | | | 1% |
|---|---|---|---|---|
| | | 1% | 1% Tea | Mulberry |
| Composition | Control | AC | polyphenols | phenols |
| Virgin | −25% | −4% | −23% | −13% |
| Bleached | −43% | −6% | −38% | −19% |

To each test formula, CapiSeal ™ was added at 3% to the SLS-based shampoo system. Results are the average of at least 10 measurements.

### Squalane Benefit

In Table 5, cells 8 and 10 show that squalane is of benefit in the composition. Without squalane the product is still quite effective, however with 4% squalene, performance is substantially improved.

TABLE 5
Change in Break Strength

| | | Test Formula Number | | |
|---|---|---|---|---|
| Treat | None | | 8 | 10 |
| Virgin | −25% | | −9% | −14% |
| Bleached | −43% | | −7% | −14% |
| Squalane | | | 4% | 0% |

To each test formula, CapiSeal ™ was added at 3% to the SLS-based shampoo system. Results are the average of at least 10 measurements.

### Aesthetic Effects

Finally the last table, Table 6, demonstrates the optimal concentration of CapiSeal™ to use in a shampoo system. The concentration of CapiSeal™ was varied, formula number 1 from Table 1, and examined the damage preventing effects at 1%, 3% and 5%. As seen in Table 6, while 1% CapiSeal™ provided modest damage minimizing effects (about half the damage observed in the control), increasing the concentration to 3% dramatically improved performance (about 90% damage prevention). Further increasing CapiSeal™ to 5% provided little additional efficacy. Therefore, we recommend 3% as the optimal concentration.

TABLE 6
Change in Break Strength-Concentration Dependence of CapiSeal ™

| CapiSeal ™ | Control-0% | 1% | 3% | 5% |
|---|---|---|---|---|
| Virgin | −28% | −19% | −5% | −4% |
| Bleached | −41% | −24% | −8% | −6% |

Results are the average of at least 10 measurements.

### No Control Study

In a no control in use preference study, we asked 30 subjects to use a simple shampoo with CapiScal™ (formula #1) at 1%, 3% or 5%. Ten subjects used a specified concentration for 7 days, shampooing daily, as their only hair grooming product.

Panelist were asked two questions at the end of the study: “Did they notice any beneficial conditioning effects?” and “Did they notice any negative build up or over-conditioning?”

No Control Results are summarized as follows:

### 1% CapiSeal™

Did they notice any beneficial conditioning effects? 8/10 yes
Did they notice any negative build up or over-conditioning? 0/10 yes

### 3% CapiSeal™

Did they notice any beneficial conditioning effects? 10/10 yes
Did they notice any negative build up or over-conditioning? 0/10 yes

### 5% CapiSeal™

Did they notice any beneficial conditioning effects? 10/10 yes
Did they notice any negative build up or over-conditioning? 2/10

### CONCLUSION

Based upon consumer testing and break strength measurements. CapiSeal™ formula #1, a preferred concentration range of 1% to 5%, with 3% being an optimal example of the present invention. The carrier triglyceride oil seems unimportant. Both sunflower and safflower oil provide similar benefits. Cost, cache and aesthetic considerations should determine which product to use. Squalane at 4% enhances product activity. Higher concentrations were not tested due to cost considerations. Vegelite 912, a permeation enhancer, works best at 10% and is superior to similar hydrocarbons. The argan concentrate is most effective at 0.1% to 1.0%. Considering cost, 0.1% is an optimal concentration to use. The argan concentrate is specific in providing benefits while similar terpenoid blends result in reduced benefits. The 3% use of CapiSeal™ in finished formula is an optimal concentration for maximizing efficacy and leading to no untoward aesthetic effects.

It is to be understood that the above description and examples are intended to be illustrative and not restrictive. Many embodiments will be apparent to those of skill in the art upon reading the above description and examples. The scope of the invention should, therefore, be determined not with reference to the above description and examples but should instead be determined with reference to the appended claims, along with the full scope of equivalents to which such claims are entitled. The disclosures of all articles and references, including patent applications and publications, are incorporated herein by reference for all purposes.

What is claimed is:

1. An oil-based composition for use in a rinse off or leave on hair product for protecting hair cuticles, the composition comprising:
a triglyceride carrier;
squalene;
a coconut oil-based alkane;
melon seed oil;
argan seed oil; and
argan seed oil unsaponifiables.

2. The composition as recited in claim 1, wherein the triglyceride carrier is sunflower oil or safflower oil.

3. The composition as recited in claim 1, wherein the coconut oil-based alkane is Vegelite 912.

4. The composition as recited in claim 1, wherein the melon seed oil contains triglycerides.

5. The composition as recited in claim 1, wherein the argan seed oil contains triglycerides.

6. The composition as recited in claim 1, wherein the argan seed oil unsaponifiables are terpenoids or branched hydrocarbons.

7. The composition as recited in claim 1, wherein the composition contains 73.9% to 93.9% of the triglyceride carrier, 0% to 20% of the coconut oil-based alkane, 0% to 4% squalene, 1% of the melon oil, 1% of the argan seed oil and 0% to 1% of the argan seed oil unsaponifiables.

8. The composition as recited in claim 7, wherein 1% to 5% of the composition is used in the rinse off or the leave on product.

9. The composition as recited in claim 7, wherein 3% of the composition is used in the rinse off or the leave on product.

10. A method for improving the condition of hair, the method comprising:
using a rinse off or leave on hair product comprising 0.001% to 25% of a composition, wherein the composition comprises a triglyceride carrier, squalene, a coconut oil-based alkane, melon seed oil, argan seed oil, and argan seed oil unsaponifiables.

11. The method as recited in claim 10, wherein the improved hair condition is hair break strength.

12. The method as recited in claim 10, wherein the improved hair condition is hair moisture content.

13. The method as recited in claim 10, wherein the improved hair condition is hair shine.

14. The method as recited in claim 10, wherein the improved hair condition is inter fiber friction.

15. The method as recited in claim 10, wherein the improved hair condition is hair curl retention.

16. The method as recited in claim 10, wherein the improved hair condition is hair smoothness.

17. The method as recited in claim 10, wherein the improved hair condition is hair elasticity.
